(19) United States
(12) Patent Application Publication
(10) Pub. No.: US 2024/0109066 A1
(43) Pub. Date: Apr. 4, 2024

(54) MICROFLUIDIC MIXING AND/OR SEPARATER

(71) Applicant: West Pharmaceutical Services, Inc., Exton, PA (US)

(72) Inventors: James G. GUTHLEIN, Malvern, PA (US); Michael J. SULLIVAN, Birdsboro, PA (US); Rajiv KUMAR, Exton, PA (US)

(21) Appl. No.: 18/376,397

(22) Filed: Oct. 3, 2023

Related U.S. Application Data
(60) Provisional application No. 63/378,230, filed on Oct. 3, 2022.

(51) Int. Cl. B01L 3/00

(52) U.S. Cl. CPC B01L 3/502746; B01L 3/502761; B01L 2300/0829; B01L 2400/088

(57) ABSTRACT

A microfluidic device may have at least one inlet channel; a microfluidic channel having a first portion fluidly connected to the at least one inlet channel; and at least one outlet channel fluidly connected to a second portion of the microfluidic channel, wherein the microfluidic channel has a plurality of dimples extending away from an axis of the microfluidic channel. The at least one inlet channel includes a first inlet channel and a second inlet channel, and/or the at least one outlet channel includes a first outlet channel and a second outlet channel. In some embodiments, the plurality of dimples may be configured to separate nanoparticles of different sizes to the first outlet channel and the second outlet channel.

### CROSS REFERENCE TO RELATED APPLICATIONS

The patent application claims the benefit of U.S. Provisional Patent App. No. 63/378,230 filed on Oct. 3, 2022, the entire disclosure of which is incorporated herein by reference.

### TECHNICAL FIELD

The disclosure relates to devices, systems, and methods of use for mixing at least two substances to produce a pharmaceutical complex and/or separating monodispersed nanoparticles.

### BACKGROUND

Recent developments in immunology include newly-approved messenger RNA-lipid nanoparticle (mRNA-LNP) vaccines. Messenger RNA (mRNA) technology has the advantage of being able to rapidly adapt to new antigen designs by altering the mRNA sequence without needing to overhaul the Chemical & Manufacturing Control (CMC) of the vaccine production. However, mRNA provided alone is not readily absorbed or delivered effectively to human immune cells and has unstable chemical and physical properties, therefore is not effective for use as a vaccine. Recent developments have shown that absorption and stability of mRNA can be increased to effective levels if it is encapsulated within lipid nanoparticle (LNP) vectors.

Preparation of mRNA-LNP vaccines is achieved by mixing lipids dissolved ethanol with RNA in buffers, under closely controlled conditions. Such mixing is usually carried out in a laboratory using devices which are often inappropriate for high-scale distribution, due to their low durability, high cost, high complexity, low lot-to-lot consistency and/or high inter-batch variation.

### SUMMARY

The present inventors recognize that the shelf life of mRNA-LNP at room temperature is limited. To extend the shelf life, mRNA-LNP vaccines must be stored at extremely low temperatures (typically −20 to −80 degrees Celsius). This is a problem because low temperature distribution is expensive and logistically complex. Additionally, there is a risk of mRNA-LNP vaccines being wasted if, for example, the low-temperature environment at any stage in the distribution chain were to fail.

Non-messenger RNA drugs, such as RNAi, siRNA and other oligonucleotides can also be formed into lipid nanoparticle compositions (RNA-LNP). RNA-LNP drugs can be chemically modified to improve their stability and shelf life at room temperature (such chemical modification is not possible for mRNA-LNP technology which requires interaction with cellular proteins to function appropriately). Chemical modification of RNA-LNP can be difficult and expensive to achieve but is nonetheless often preferred to avoid the significant distribution costs associated with non-modified RNA-LNP drugs which must similarly be kept at very low temperatures, as well as the difficulty associated with managing drug efficacy over time due to the limited molecular half-life.

In short, the low temperature requirements present a major challenge for distribution and development. Other problems associated with known systems for producing nanoparticle compositions include limited scalability, usability, and/or reliability. One or more of the foregoing needs are met by the various embodiments as disclosed herein.

A first aspect of the present disclosure is directed to a microfluidic device having at least one inlet channel; a microfluidic channel having a first portion fluidly connected to the at least one inlet channel; and at least one outlet channel fluidly connected to a second portion of the microfluidic channel, wherein the microfluidic channel has a plurality of dimples extending away from an axis of the microfluidic channel.

The microfluidic device may include one or more of the following features. The at least one inlet channel may include a first inlet channel and a second inlet channel. The at least one outlet channel may include a first outlet channel and a second outlet channel. The plurality of dimples may be arranged circumferentially around the microfluidic channel. The plurality of dimples may be arranged in sets that longitudinally overlap. The plurality of dimples may include a first set of dimples arranged longitudinally along the microfluidic channel and a second set of dimples arranged longitudinally along the microfluidic channel, the first set of dimples and the second set of dimples are laterally offset, and the first set of dimples and the second set of dimples are configured to separate nanoparticles by size. The first set of dimples may have a first width or diameter, the second set of dimples have a second width or diameter of a second size, and the first width or diameter and the second width or diameter are different. The first width or diameter may be about 50 μm to about 200 μm, and the second width or diameter may be about 200 μm to about 500 μm. The at least one outlet channel may include a first outlet channel and a second outlet channel, the first set of dimples may be arranged to guide nanoparticles of a first size to the first outlet channel, and the second set of dimples may be arranged to guide nanoparticles of a second size to the second outlet channel. The at least one outlet channel may include a third outlet channel, and the plurality of dimples may include a third set of dimples arranged longitudinally along the microfluidic channel. The third set of dimples may be configured to guide nanoparticles of the second size to the third outlet channel.

A second aspect of the disclosure is directed to a microfluidic device having: a plurality of inlet channels; a microfluidic channel having a first portion fluidly connected to the plurality of inlet channels; and a plurality of outlet channels fluidly connected to a second portion of the microfluidic channel, wherein the microfluidic channel has a plurality of dimples extending away from an axis of the microfluidic channel.

The microfluidic device may include one or more of the following features. The plurality of outlet channels may include a first outlet channel, a second outlet channel, and a third outlet channel. The plurality of dimples includes a first set of dimples arranged to guide nanoparticles of a first size to the first outlet channel, a second set of dimples arranged to guide nanoparticles of a second size to the second outlet channel, and a third set of dimples arranged to guide nanoparticles of the second size to the second outlet channel.

A third aspect of the disclosure is directed to a method of manufacturing a microfluidic channel. The method may include injecting one or more elastomeric materials into a mold cavity around a core pin, wherein the core pin has an elongated shaft with a plurality of protrusions extending from an axis of the core pin; forming a component including a microfluidic channel with a plurality of dimples from the one or more elastomeric materials; and removing the core pin from the component.

The method may include one or more of the following features. The method may further include removing the component and central core from the mold cavity prior to removing the core pin from the component. The one or more elastomeric materials may include a silicone, a rubber, and/or a thermoplastic elastomer. Removing the core pin from the component may be conducted with a compressed air ejector system. Removing the core pin from the component may be conducted by sliding the component off of the core pin. The protrusions may be spherical.

### BRIEF DESCRIPTION OF THE DRAWINGS

Specific embodiments of the present disclosure are described below in the detailed description by way of example only and with reference to the accompanying drawings, in which:

FIG. 1 illustrates a side view of an exemplary system according to the present disclosure.

FIG. 2 illustrates an exemplary microfluidic channel of a microfluidic device of FIG. 1.

FIG. 3 illustrates an exemplary method of manufacturing the microfluidic device of FIGS. 1 and 2.

FIG. 4 illustrates an exemplary core pin used in the method of FIG. 3.

FIG. 5 illustrates a second embodiment of the microfluidic device of FIG. 1.

The same reference numbers are used in the drawings and the following detailed description to refer to the same or similar parts.

### DETAILED DESCRIPTION

A microfluidic device is provided for connecting one or more fluid containers for mixing first and second substances to produce a pharmaceutical complex. The microfluidic device may include a first port or connector member configured to connect to a first container, a second port or connector member configured to connect to a second container, and a third port or connector member configured to connect to a recipient container. The microfluidic device may further include a microfluidic channel extending from a first portion in fluid communication with the first port and the second port to a second portion in communication with the third port. The microfluidic device may include obstacles configured to generate chaotic mixing of fluids, for example, to allow charged nanoparticles to self-assemble in a predetermined alignment and structure based on their chemical make-up, charge, and/or shape. The obstacles may be produced by dimples in the flow channel that create eddy channels with the flow path when a specific flow rate is achieved. The size, shape, orientation, and patterning of the obstacle geometry may be specifically and purposefully designed to generate flow paths with different chaos and turbulence for different applications. In some embodiments, the microfluidic device may, additionally or alternatively, be configured to separate the formed nanoparticles by size due to obstacle geometry of the channel. The microfluidic device may produce targeted turbulent flow produced by the obstacle geometry, where different size nanoparticles will be moved to specific flow regions of the microfluidic channel, essentially separating the nanoparticles by size due to the flow characteristics of the channel.

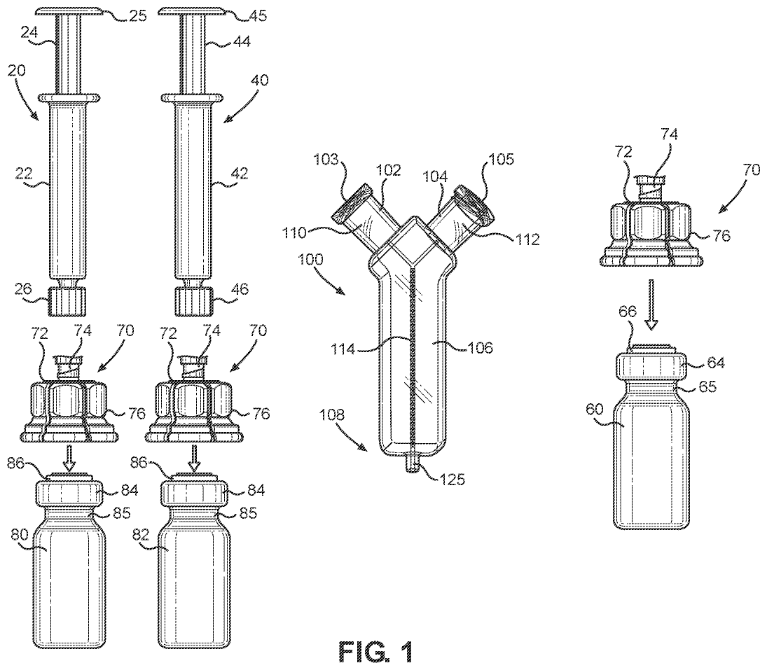

FIG. 1 illustrates a system including a first container 20, a second container 40, a recipient container 60, a microfluidic device 100. In some embodiments, the system may further include a first storage container 80 containing a first substance or constituent and a second storage container 82 containing a second substance or constituent. The first container 20 may be configured to transfer the first substance from the first storage container 80 into the microfluidic device 100. The second container 40 may be configured to transfer the second substance from the second storage container 82 into the microfluidic device 100. The microfluidic device 100 may include at least one path configured to mix the first substance and the second substance and transfer the pharmaceutical complex to the recipient container 60. The system may constitute a kit of at least one or all of the first container 20, the second container 40, the recipient container 60, the microfluidic device 100, the first storage container 80, and/or the second storage container 82. The system and/or kit may further include one or more vial adapters 70 for fluid transfer to or from one or more of the recipient container 60, the first storage container 80, and/or the second storage container 82. The components of the kit may include packaging for transportation to the end user.

The first container 20 may be a variable volume container, such as a first syringe configured to at least temporarily store and/or transfer the first substance from the first storage container 80 to the microfluidic device 100. The second container 40 may be a variable volume container, such as a second syringe configured to store and transfer the second substance from the storage container 82 to the microfluidic device 100. The first syringe 20 may include a first syringe body 22 and a first plunger rod 24, and the second syringe 40 may include a second syringe body 42 and a second plunger rod 44. Each syringe body 22, 42 may have a syringe barrel extending from a proximal end to a distal end along a longitudinal direction. Each syringe body 22, 42 may have a syringe tip at the distal end and a flange at the proximal end. The syringe barrel may be tubular having an inner surface extending along the longitudinal direction to define a chamber. The chamber may be configured to receive, store, and/or mix the substance for dispensing through a distal opening of the syringe tip. The first plunger rod 24 may have a first flange 25 at a proximal end, and the second plunger rod 44 may have a second flange 45 at a proximal end. The syringe tip of the first syringe body 22 may include a first connector 26 for engagement with an external device, such as a syringe needle, a container, and/or the microfluidic device 100. The syringe tip of the second syringe body 42 may include a second connector 46 for engagement with the same or different external device, such as a syringe needle, a container, and/or the microfluidic device 100. Each connector 26, 46 may further include a male Luer connector including the syringe tip and a threaded sleeve around the tip. The syringe tip may be tapered to guide fluid flow into the external device (e.g., the microfluidic device 100), and the sleeve may have an internal thread configured to secure the syringe 20, 40 to the respective external device (e.g., the microfluidic device 100). The containers 20, 40 may be any conventional type of syringe and/or reciprocating pump which is suitable for use in a pharmaceutical setting.

The flange 25, 45 may be actuated either by being pulled to create a negative pressure to pull a substance into the chamber and/or being pushed to create a positive pressure to push the substance out of the chamber. At least part of the first syringe 20 and the second syringe 40 may be integrally or releasably connected to enable joint handling and/or actuation of the first syringe 20 and the second syringe 40. For example, the system may further have a barrel holder (not shown) having a first lumen configured to receive the first syringe body 22 and a second lumen configured to receive the second syringe body 42, such that the first syringe 20 and the second syringe 40 may be handled together. Each of the first and second lumens may be closed or be formed by C-shaped walls configured to snap around the respective syringe body 22, 42. The barrel holder may fix the syringe bodies 22, 42 in a substantially parallel arrangement. The system may further include a plunger clip configured to translate the plunger rods 24, 44 through the syringe bodies 22, 42 together to push and/or pull the material with the same longitudinal translation. For example, the plunger clip may be configured to attach to the flanges 25, 26, such as having a groove configured to releasably receive the flanges 25, 26. Embodiments of the barrel holder and/or the plunger clip are further discussed in U.S. Pat. Nos. 5,104,375, 6,840,921, and 8,240,511, the entire disclosures of which are incorporated herein by reference.

The recipient container 60 may be a fixed volume container, such as a vial attachable to the microfluidic device 100 with a vial adapter 70. The vial 60 may include a vial bottle 62 enclosing a chamber and having a crown and a neck. The chamber may be sealed by a drug vial seal at the crown attached circumferentially by an aluminum band. The vial adapter 70 may have a transverse top wall 72, a connector 74 extending upwardly from the top wall 72, and a skirt 76 extending downwardly from the top wall 72. The connector 74 may be a female Luer connector including an external screw thread for screw thread engagement by a male Luer lock connector, such as that of the microfluidic device 100. The skirt 76 may be for telescopic mounting over the crown and/or the neck of the vial 60. The skirt 76 may surround a cannula (not shown) extending downwardly from the top wall 72 and be configured to puncture the vial stopper. The cannula may have a lumen in fluid communication with the chamber of the vial bottle 62 when puncturing the vial stopper. The vial adapter 70 may be vented in order to draw air into the container 60 and to ease drawing the fluid through the system. Further discussion of embodiments of the container 60 and/or the vial adapters 70 is provided in U.S. Pat. Nos. 8,753,325 and 9,943,463, the entire disclosures of which are expressly incorporated herein by reference. The recipient container 60 may be initially empty and be configured to receive material injected from the first and second containers 20, 40 and mixed in the microfluidic device 100. Once the first and second constituents are introduced into the microfluidic device 100, the resultant pharmaceutical complex may be stored within the recipient container 60.

However, in some embodiments, the recipient container 60 may be a variable volume container, such as a syringe, and one or both of the first and second containers 20, 40 may be fixed volume containers, such as vials. Further discussion of such embodiments is provided in U.S. Pat. Pub. 2023/0105059, the entire disclosure of which is expressly incorporated herein by reference.

The first storage container 80 and/or the second storage container 82 may have similar structure as the recipient container 60, the discussion of which is expressly incorporated herein in its entirety. For example, each of the first and second storage containers 80, 82 may be a fixed volume container, such as an enclosing a chamber and having a crown 84 and a neck 85. The chamber may be sealed by a drug vial seal 86 at the crown 84 attached circumferentially by an aluminum band. Each of the first and second storage containers 80, 82 may be attached to a vial adapter 70, as discussed with reference to the recipient container 60.

The first substance of the first storage container 80 may be an aqueous solution. The aqueous solution may be any aqueous buffers which may be used for dissolving nucleic acids. For example, in some embodiments, the aqueous solution may be a solution of 20 mM Citrate and 300 mM NaCl and have a pH in the range of 3 to 6. In some embodiments, the aqueous solution may be 20 mM phosphate buffer solution (PBS) at pH 7. In some embodiments, the aqueous solution may be a solution of 5 mM to 25 mM Sodium acetate buffer with pH range from 4 to 6.

The second substance of the second storage container 82 may be a lipid solution having a composition comprising in whole, or in part, an organic solvent having a lipid or mixture of lipids. The lipid solution may include clinical grade lipids solubilized in an organic alcohol solution (e.g., ethanol). In some embodiments, the lipid solution may be at least 25% alcoholic solution. In some embodiments, the lipid solution may be at least 40% alcoholic solution. In some embodiments, the lipid solution may be at least 60% alcoholic solution. The alcoholic solution is preferably an ethanolic solution. Providing the lipid in an increased concentration of alcohol (e.g., greater than 40% alcoholic solution) may allow the lipid in alcoholic solution to withstand dilution by a reconstituting agent without affecting the quality of the resulting pharmaceutical complex. The lipids compositions in ethanol solution may be composed of an ionizable lipid or cationic lipids or synthetic lipids, structural lipids, a PEG-lipid or its derivative and cholesterol or its derivatives. However, the second substance may include other nanoparticle forming solutions.

The therapeutic agent may be carried in at least one of the first substance and/or the second substance. In a preferred embodiment, the therapeutic agent is carried in the first substance. The therapeutic agent may include a nucleic acid including gene editing complexes, a drug, a protein, oligonucleotides or the like. The nucleic acid may include RNA and/or DNA. The RNA may be in the form of oligonucleotide RNA, tRNA (transfer RNA), snRNA (small nuclear RNA), rRNA (ribosomal RNA), mRNA (messenger RNA), antisense RNA, siRNA (small interfering RNA), shRNA (short-hairpin RNA), ncRNA (non-coding RNA), aptamers, ribozymes, chimeric sequences, or derivatives of these groups. Gene editing complexes may include gRNA (guide RNA), cas 9 protein, mRNA or DNA encoding for cas 9 protein or the CRISPR-cas9 gRNA complex. The DNA may be in the form of antisense, plasmid DNA, parts of a plasmid DNA, pre-condensed DNA, product of a polymerase chain reaction (PCR), vectors (P1, PAC, BAC, YAC, artificial chromosomes), expression cassettes, chimeric sequences, chromosomal DNA, or derivatives of these groups.

In some embodiments, the therapeutic agent may be stored in a dehydrated and/or lyophilized state and be reconstituted in the aqueous solution to form the first substance prior to being introduced into the microfluidic device 100. In this case, the first storage container 80 may hold the dehydrated therapeutic agent, and the first container 20 may hold the aqueous solution. The aqueous solution may then be introduced from the first container 20 into the first storage container 80 to reconstitute the therapeutic agent. The first solution including the therapeutic agent may then be introduced into the first container 20 to be introduced into the adapter.

The first substance (with the therapeutic agent in the lyophilized state or as a solution) and the second substance may be adapted for transportation and medium-term or long-term storage at room temperature. As such, the system and microfluidic device 100 disclosed herein may enable the obstacles associated with storing and transporting RNA-LNP complexes at prohibitively low temperatures to be mitigated. Furthermore, the microfluidic device 100 may be easy to use at the point of care.

The first substance and the second substance may be mixed with the microfluidic device 100 as discussed herein. The mixing may generate liposomal formations that entrap the therapeutic agent coincident with formation of the liposomes. An electrostatic interaction between the negatively charged therapeutic agent (e.g., nucleic acid) and positively charged cationic lipid may bring about the encapsulation, forming the pharmaceutical complex. The pharmaceutical complex may be monodispersed Lipid Nano Particles (LNP). Accordingly, the system and microfluidic device 100 may be used to form a ready-to-inject RNA-LNP (for instance, mRNA-LNP) complex by mixing the constituents of the aqueous solution including RNA and the lipid solution. Considering the specific example of forming the mRNA-LNP pharmaceutical complex, the complex may be formed by mixing the first substance including mRNA (or RNA) from the first container 20 and the second substance of the lipid solution from the second container 40.

The first and second storage containers 80, 82 may be the same or different sizes based on the intended mixture. Any reference to two storage containers 80, 82 herein should therefore be construed as including three or more storage containers 80, 82. It will be appreciated that if more than two constituents are to be mixed then more than two containers may be provided, each of which may contain at least one constituent. Furthermore, any number of constituents can be provided in an unmixed state within a single container. In some embodiments, the first container 20 and the second container 40 may be prefilled with the respective substance, for example embodied as prefilled syringes, such that the first and second storage containers 80, 82 may be omitted.

The microfluidic device 100 may define a first port 102 configured to connect to the syringe connector 26 of the first container 20 and a second port or connector member 104 configured to attach to the syringe connector 46 of the second container 40. The first port 102 and the second port 104 may be connected to a first upper portion of a mixing member 106. A third port or connector member 108 may be connected at a second bottom portion of the mixing member 106.

The first port 102 may be configured to be received by the first syringe connector 26 and have an external thread 103 configured to threadably engage the internal threads of the first connector 26. Similarly, the second port 104 may be configured to be received by the second connector 46 and have an external thread 105 around the second tubular member 108 configured to threadably engage the internal threads of the second syringe connector 46. For example, the ports 102, 104 may be a female Luer connector, and the connectors 26, 46 of the containers 20, 40 may be a male Luer connector. However, the ports 102, 104 may, additionally or alternatively, connect to the containers 20, 40 with other types of connections such as a snap-fit, friction-fit and/or a press-fit. The microfluidic device 100 may be configured to connect to any number of the first and second containers 20, 40, the microfluidic device 100 may have an equal number of ports 102, 104 for connecting to each of the containers 20, 40 accordingly. Furthermore, one or more of the ports 102, 104 may include a one-way valve (not shown) to allow fluid flow from the container 20, 40 into the microfluidic device 100, and to restrict or substantially prevent fluid flow out of the microfluidic device 100 back to the respective container 20, 40.

The first port 102 may define a first inlet channel 110, and the second connector member 104 may define a second inlet channel 112. In some embodiments, the first inlet channel 110 may be configured to receive the tip of the first syringe 20 to place the chamber of the first syringe body 22 in fluid communication with the first inlet channel 110, and the second inlet channel 112 may be configured to receive the tip of the second syringe 40 to place the chamber of the second syringe body 42 in fluid communication with the second connector channel 112. In some embodiments (not shown), the first port 102 and the second port 104 may extend substantially parallel to each other to facilitate joint actuation of the syringes 20, 40. The first inlet channel 110 and the second inlet channel 112 may extend at an angle to the mixing member 106. The angle may be at least 90 degrees, and in some embodiments, may be between about 120 degrees to about 160 degrees. For example, the mixing member 106, the first port 102, and the second port 104 may form a Y-shaped portion of the microfluidic device 100. The mixing body 106 may define a microfluidic channel 114.

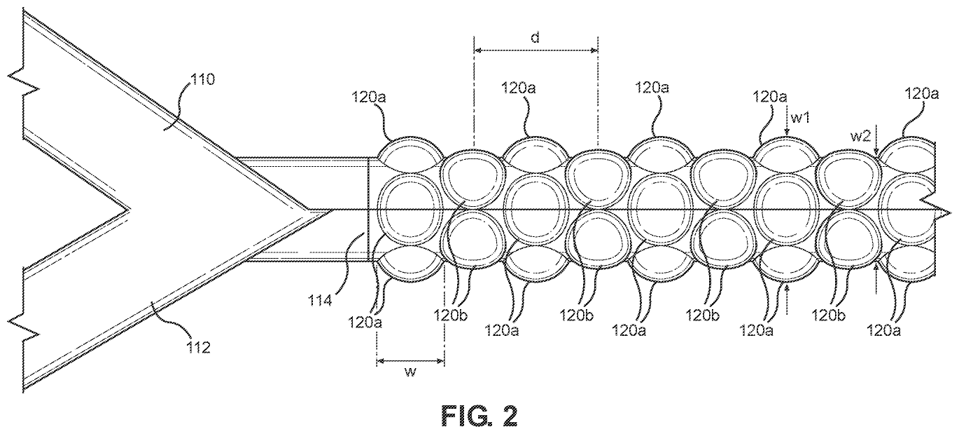

FIG. 2 illustrates an embodiment of the microfluidic channel 114. The microfluidic channel 114 may include a pathway extending along a longitudinal axis of the mixing body 106. The pathway of the microfluidic channel 114 may include a plurality of dimples 120 formed in the mixing body 106 of the microfluidic device 100. The microfluidic channel 114 may extend along a longitudinal axis of the microfluidic body 106, and the dimples 120 may extend radially outwardly from the microfluidic channel 114 and be recessed in the mixing body 106. Accordingly, to this configuration, the pathway of the fluid flowing through the microfluidic channel 114 may be tortuous extending into and out of the dimples 120. The tortuous path may meander from the longitudinal axis of the mixing body 106. This fluid obstacle geometry configuration defined by the dimples 120 may create a purposefully chaotic flow inducing eddies and turbulence to mix substances of the fluids to improve mixing and generate consistently sized pharmaceutical complexes

As illustrated in FIG. 2, the dimples 120 may be continuously disposed around a perimeter and along a longitudinal axis of the microfluidic channel 114 in a three-dimensional manner. Thus, the dimples 120 may be formed surrounding (e.g., above, below, to the sides, circumferentially around, etc.) the microfluidic channel 114, allowing the full use of the microfluidic channel 114 to perform the mixing. In some embodiments, the dimples 120 may be formed along an entire length of the microfluidic channel 114. The configuration may increase the efficiency over two-dimensional channels and provides a predictable fluid flow through the microfluidic channel 114. The size, shape, orientation, location, and pattern of this fluid obstacle geometry configuration may be tuned to allow for specific chaotic mixing that yields a predictive pattern of flow. The dimples 120 may be arranged circumferentially around the microfluidic channel 114 in sets that are longitudinally staggered or angularly offset, such that longitudinally adjacent dimples 120 are not aligned along their central axes. The sets may be formed by a first set of dimples 120a and a second set of dimples 120b that are rotationally offset and alternate along the longitudinal axis of the microfluidic channel 114. The staggering allows for the adjacent dimples 120 to longitudinally approximated and/or overlap increasing the density of the dimples 120. The dimples 120 may have a circular cross-section and/or have a width or diameter w of about 200 μm to about 500 μm. For example, the dimples 120 may be circular with a diameter between about 280 μm and about 325 μm. The width or diameter of the dimples 120 may be substantially uniform based on manufacturing tolerances. Longitudinally aligned dimples 120 may be separated by a distance d (from center to center) of about 500 μm to about 800 μm. The variable width or diameter of the microfluidic channel 114 may be variable due to the dimples 120. The microfluidic channel 114 may have a shorter width w1 of about 400 μm to about 600 μm and a larger width w2 of about 600 μm to about 800 μm. Further discussion of embodiments of the dimples is provided in U.S. Pat. Pub. 2023/0105059, as previously incorporated herein by reference.

Returning to FIG. 1, the microfluidic device 100 may have the third port or connector member 108 at the bottom portion of the mixing member 106. In some embodiments, the third connector member 108 may include a tip configured to attach to the recipient container 60 via the vial adapter 70. In some embodiments, the third connector member 108 may have a sleeve be in the form of a male Luer connector (not shown). The tip may have the outlet channel 125 in communication with a second or bottom portion of the microfluidic channel 114. The tip may be received in the connector 74 of the vial adapter 70 and the sleeve may be threadedly connected to an outer surface of the connector 74 in a Luer connection. The third connector member 108 may be a male Luer connector configured to connect to a female Luer connector of the vial adapter 70. However, the connector member 108 may, additionally or alternatively, connect to the third container 60 with other types of connections such as a snap-fit and/or a press-fit. The outlet channel 125 may provide passage of the mixed composition from the mixing chamber 124 to the recipient container 60.

The microfluidic device 100 may be formed of a polymer, a metal, and/or a glass. In a preferred embodiment, the microfluidic device 100 may be formed in a single, unitary piece (for example through injection molding or 3D printing) including the first port 102, the second port 104, the mixing member 106, the tubular member 129, and/or the connector member 108. Alternatively, the microfluidic device 100 may be formed of two pieces (e.g., halves) and be secured or fused together, each of which can be a metal, a polymer, or a glass. To increase the ease with which the fluid to be mixed flows through the microfluidic device 100, low surface energy materials may be used for at least a portion of the microfluidic device 100. For example, in some embodiments, the microfluidic device 100 may be formed of or coated with a low surface energy material such as ethylene tetrafluoroethylene (ETFE). Other low surface energy materials may also be used, for example fluoropolymer materials other than ETFE. Alternatively, the at least one path may be treated to reduce the surface energy. Forming the sides of the microfluidic channels 114 of low surface energy materials can reduce loss of constituents across the microfluidic channel 114 during use and therefore may enable the microfluidic device 100 to operate more efficiently. Although a low surface energy material may provide additional advantages in some embodiments, it is an optional feature of the present disclosure. In some embodiments, as discussed below, the microfluidic device 100 may, additionally or alternatively, be formed of an elastomer, such as a silicone, a rubber, and/or a thermoplastic elastomer.

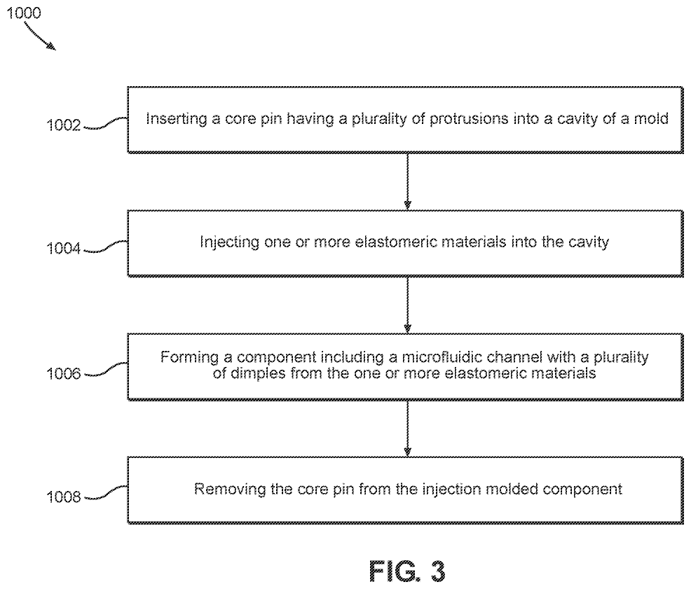

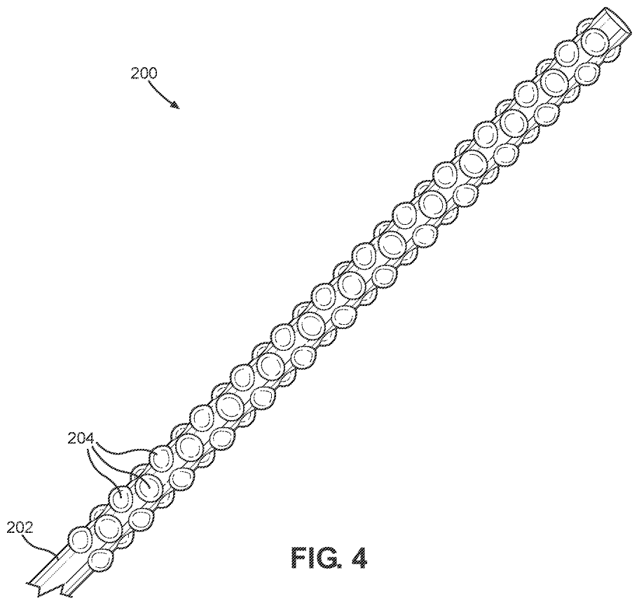

FIG. 3 illustrates a method 1000 of forming the microfluidic device 100 through injection molding, and FIG. 4 illustrates an embodiment of a core pin 200 that may be used in the method 1000. The method 1000 and the core pin 200 may address shortcomings in the production of three-dimensional internal components, such as the microfluidic channel 114 of the microfluidic device 100. The method 1000 may produce the microfluidic device 100 in a single, unitary piece through injection molding. The method 1000 may eliminate limitations in the manufacturing by utilizing an elastomeric material for at least one of the core pin 200 and/or the microfluidic device 100 to facilitate removal of the core pin 200 during the production of the unique design geometry.

In step 1002, the core pin 200 may be inserted into a cavity of a mold (not shown). The mold may be formed of two housing members releasably attached and forming the cavity. As illustrated in FIG. 4, the core pin 200 may have an elongated shaft 202 having a plurality of protrusions 204 on an outer surface of the elongated shaft 202. The protrusions 204 may have a spherical shape and be arranged along the elongated shaft 202 corresponding to the desired arrangement of the dimples 120. In some embodiments, the core pin 200 may be made of an elastomer, such as a silicone, a rubber, and/or a thermoplastic elastomer.

In step 1004, one or more material may be injected in liquid form into the cavity and around the core pin 200. In some embodiments, the one or more materials may include an elastomer, such as a silicone, a rubber, and/or a thermoplastic elastomer. The one or more materials may be molten when injected to conform around the core pin 200 inside of the cavity.

In step 1006, a component may be formed including a microfluidic channel with a plurality of dimples from the one or more materials. The component may be formed by cooling the one or more materials. The one or more materials may crosslink and/or vulcanize to form the component, such as the microfluidic device 100. The one or more materials may form the microfluidic channels 114 along the shaft 202 with the dimples 120 being formed around the protrusions 204 to form the desired obstacle geometry.

In step 1006, the injection molded component, such as the microfluidic device 100, may be removed from the cavity of the mold. The core pin 200 may be removed from the microfluidic channel 114 of the microfluidic device 100. The flexibility of the elastomeric material of the microfluidic device 100 and/or the core pin 200 may allow extraction of the core pin 200 from the microfluidic device 100 without stripping the dimples 120 from the microfluidic channel 114. In some embodiments, the core pin 200 may be removed from the microfluidic device 100 with an air ejector system in the molding assembly that will apply pressure to release the core pine 200 from the microfluidic device 100.

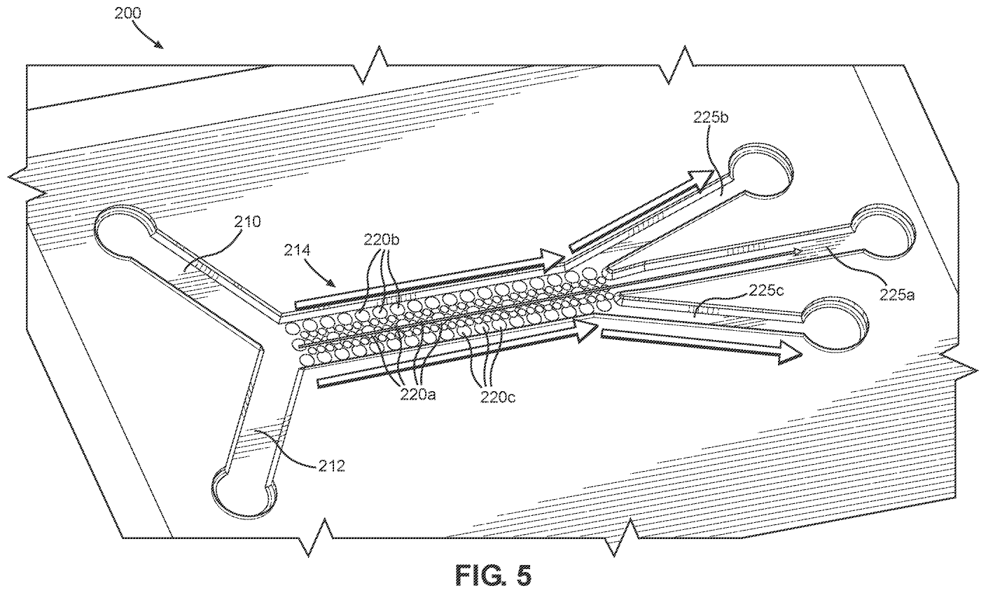

FIG. 5 illustrates a second embodiment of a microfluidic device 200 that may be implemented in the system 10. The microfluidic device 200 may include a first inlet channel 210 configured to a receive a first substance or constituent and a second channel 212 may be configured to receive a second substance or constituent. In some embodiments, the first substance may be an aqueous solution and the second substance may be a lipid solutions, as discussed with reference to the system and the microfluidic device 100 as expressly incorporated herein by reference in its entirety.

The microfluidic device 200 may include a microfluidic channel 215 having a first portion in communication with the first inlet channel 210 and the second channel 212. The microfluidic channel 215 may be configured to mix the first substance and the second substance and/or separate the formed nanoparticles by size. The microfluidic channel 215 may have a plurality of obstacles 220 arranged to direct multiple and specific chaotic flow streams in the microfluidic channel 215. The flow streams may be generated by a variable arrangement of the obstacles 220, forcing larger nanoparticles along one or more first fluid paths and smaller nanoparticles along one or more second fluid paths. The plurality of obstacles 220 may include a first set of obstacles 220a arranged longitudinally along a first fluid path of the microfluidic channel 215. The plurality of obstacles 220 may include a second set of obstacles 220b arranged longitudinally along a first second path of the microfluidic channel 215. The plurality of obstacles 220 may include a third set of obstacles 220b arranged longitudinally along a third second path of the microfluidic channel 215.

In some embodiments, the first set of obstacles 220a may be separated by a first distance and the second set of obstacles 220b may be separated by a second distance, where the first distance is different than the second distance. The third set of obstacles 220c may be separated by a third distance, where the third distance may be the same as the second distance. In some embodiments, the first set of obstacles 220a may have a first width or diameter, and the second set of obstacles 220b may have a second width or diameter, where the first width or diameter is different than the second width or diameter. The third set of obstacles 220c may have a third width or diameter, where the third width or diameter may be the same as the third distance. In some embodiments, the first set of obstacles 220a may have a first depth, and the second set of obstacles 220b may have a second depth, where the first depth is different than the second depth. The third set of obstacles 220c may have a third depth, where the third depth may be the same as the third depth. Thus, the obstacle geometry of the obstacles 220 may generally force larger nanoparticles along one or more fluid paths and smaller nanoparticles along one or more different fluid paths.

As further illustrated in FIG. 5, the first set of obstacles 220a may be arranged on a central portion along a central line or axis of the channel 214 and be configured to guide nanoparticles to a first outlet channel 225a. The second set of obstacles 220b may be arranged on a first lateral portion of the channel 214 and be configured to guide nanoparticles to a second outlet channel 225b. The second set of obstacles 220b may be arranged on a second lateral portion of the channel 214 and be configured to guide nanoparticles to a third outlet channel 225c. The second set of obstacles 220b and the third set of obstacles 220c may be on opposite lateral sides of the first set of obstacles 220a.

In some embodiments, the first width or diameter may be less than the second width or diameter and/or the third width or diameter. For example, the first width or diameter may be about 50 μm to about 200 μm, and the second width or diameter and/or the third with or diameter may be about 200 μm to about 500 μm. Additionally or alternatively, the first distance may be less than the second distance and/or the third distance. Additionally or alternatively, the third depth may be less than the second depth and/or the third depth. Thus, in some embodiments, the obstacle geometry of the channel 214 may keep smaller particles in the center of the channel 214 guided into the first outlet channel 225a and forcing larger particles to the outer edges of the channel 214 guided into the second outlet channels 225b. The obstacle geometry may separate particles by size to generate a high-quality collectable yield of particles of desired size.

The size and configuration of the obstacles 220 may be designed based on the desired size and source of nanoparticles. In some embodiments, the first width or diameter may be greater than the second width or diameter and/or the third width or diameter. Additionally or alternative, the first distance may be greater than the second distance and/or the third distance. Additionally or alternative, the third depth may be greater than the second depth and/or the third depth. In some embodiments, the third set of obstacles 220c may be different than the second set of obstacles 220b. In some embodiments, the third set of obstacles 220c and/or the third outlet channel 225c may be omitted when two fluid paths are desired.

The microfluidic channel 215 may have a substantially rectangular cross-section, having a pair of long sides extending the width of the microfluidic channel 215 and a pair of short sides extending the height of the microfluidic channel 215. The obstacles 220 may extend to or from a planar bottom surface of the microfluidic channel 215. In some embodiments, the obstacles 220 may extend from a plurality of surfaces of the microfluidic channel 215, for example, the obstacles may extend to or from the planar bottom surface formed by a first long side and a planar top surface formed by a second long side of the substantially rectangular cross-section.

In some embodiments, the obstacles 220 may be dimples extending from the microfluidic channel 215, as further discussed regarding the microfluidic device 100 as expressly incorporated herein by reference. In some embodiments, the obstacles 220 may be projections extending into the microfluidic channel 215.

As further illustrated, the microfluidic device 200 may be in the form of or be included in a microfluidic chip. In some embodiments, the microfluidic chip may be formed by a two-piece housing joined by one or more removable fasteners, such as screws, nuts, bolts, clips, straps, and/or pins. For example, the microfluidic channel 114 may be formed in or both of the components of the two-piece housing. However, the microfluidic channel 114 may be formed in a separate microfluidic structure or plate received between the two-piece housing to seal the microfluidic structure therebetween. One or more inlet ports 102, 104 and/or one or more outlet ports 108 (as illustrated with respect to the microfluidic device 100) may be formed in the housing. In some embodiments, the microfluidic device 200 may be a single, unitary piece (for example formed through injection molding or 3D printing). The adapter 200 may be made or formed similar to the microfluidic device 100, as expressly incorporated herein by reference.

The microfluidic channel 215 may be configured to mix the first substance and the second substance to form the nanoparticles and separate the nanoparticles, such that the first inlet channel 210 and the second channel 212 may be directly connected to the microfluidic channel 215 as illustrated in FIG. 5. However, in some embodiment, the nanoparticles may be formed by a second channel, such as the microfluidic channel 114, fluidly connected to the microfluidic channel 215. Thus, the system may include a first microfluidic channel having a first cross-section for mixing (as illustrated with respect to the microfluidic channel 114) and a second microfluidic channel having a second cross-section for separating the formed nanoparticles (as illustrated with respect to the microfluidic channel 215). The inlet ports 210, 212 may be directly connected to the microfluidic channel 115, and the first microfluidic channel 115 and the second microfluidic channel 215 may be connected by a single connecting channel. For example, the first microfluidic channel 115 may have circumferentially disposed dimples to mix the first and second substances to form the nanoparticles, and the second microfluidic 215 may have laterally disposed dimples to separate the formed nanoparticles. The microfluidic channel 114 and the microfluidic channel 215 may be in the same chip or different chips. In some embodiments when on different chips, the chips may have different inlets and/or outlets.

It will also be appreciated by those skilled in the art that modifications can be made to the example embodiments described herein without departing from the invention. Structural features of systems and apparatuses described herein can be replaced with functionally equivalent parts or omitted entirely. Moreover, it will be appreciated that features from the embodiments can be combined with each other without departing from the disclosure.

What is claimed is:

1. A microfluidic device comprising:
at least one inlet channel;
a microfluidic channel having a first portion fluidly connected to the at least one inlet channel; and
at least one outlet channel fluidly connected to a second portion of the microfluidic channel,
wherein the microfluidic channel has a plurality of dimples extending away from an axis of the microfluidic channel.

2. The microfluidic device of claim 1, wherein the at least one inlet channel includes a first inlet channel and a second inlet channel.

3. The microfluidic device of claim 1, wherein the at least one outlet channel includes a first outlet channel and a second outlet channel.

4. The microfluidic device of claim 1, wherein the plurality of dimples are arranged circumferentially around the microfluidic channel.

5. The microfluidic device of claim 4, wherein the plurality of dimples are arranged in sets that longitudinally overlap.

6. The microfluidic device of claim 1, wherein the plurality of dimples includes a first set of dimples arranged longitudinally along the microfluidic channel and a second set of dimples arranged longitudinally along the microfluidic channel, the first set of dimples and the second set of dimples are laterally offset, and the first set of dimples and the second set of dimples are configured to separate nanoparticles by size.

7. The microfluidic device of claim 6, wherein the first set of dimples have a first width or diameter, the second set of dimples have a second width or diameter of a second size, and the first width or diameter and the second width or diameter are different.

8. The microfluidic device of claim 7, wherein the first width or diameter is about 50 μm to about 200 μm, and the second width or diameter is about 200 μm to about 500 μm.

9. The microfluidic device of claim 6, wherein the at least one outlet channel includes a first outlet channel and a second outlet channel, the first set of dimples is arranged to guide nanoparticles of a first size to the first outlet channel, and the second set of dimples is arranged to guide nanoparticles of a second size to the second outlet channel.

10. The microfluidic device of claim 9, wherein the at least one outlet channel includes a third outlet channel, and the plurality of dimples includes a third set of dimples arranged longitudinally along the microfluidic channel.

11. The microfluidic device of claim 10, wherein the third set of dimples is configured to guide nanoparticles of the second size to the third outlet channel.

12. A microfluidic device comprising:
a plurality of inlet channels;
a microfluidic channel having a first portion fluidly connected to the plurality of inlet channels; and
a plurality of outlet channels fluidly connected to a second portion of the microfluidic channel,
wherein the microfluidic channel has a plurality of dimples extending away from an axis of the microfluidic channel.

13. The microfluidic device of claim 12, wherein the plurality of outlet channels includes a first outlet channel, a second outlet channel, and a third outlet channel.

14. The microfluidic device of claim 12, wherein the plurality of dimples includes a first set of dimples arranged to guide nanoparticles of a first size to the first outlet channel, a second set of dimples arranged to guide nanoparticles of a second size to the second outlet channel, and a third set of dimples arranged to guide nanoparticles of the second size to the second outlet channel.

15. A method of manufacturing a microfluidic channel, the method comprising:
injecting one or more elastomeric materials into a mold cavity around a core pin, wherein the core pin has an elongated shaft with a plurality of protrusions extending from an axis of the core pin;
forming a component including a microfluidic channel with a plurality of dimples from the one or more elastomeric materials; and
removing the core pin from the component.

16. The method of claim 15, further comprising removing the component and central core from the mold cavity prior to removing the core pin from the component.

17. The method of claim 15, wherein the one or more elastomeric materials may include a silicone, a rubber, and/or a thermoplastic elastomer.

18. The method of claim 15, wherein the removing the core pin from the component is conducted with a compressed air ejector system.

19. The method of claim 15, wherein the removing the core pin from the component is conducted by sliding the component off of the core pin.

20. The method of claim 15, wherein the protrusions are spherical.
